(19) United States
(12) Patent Application Publication
(10) Pub. No.: US 2002/0165281 A1
(43) Pub. Date: Nov. 7, 2002

(54) Treatment of anoxic encephalophathy by administration of N-acetylcystein

(76) Inventor: Assa Weinberg, Los Angeles, CA (US)

Correspondence Address: Dave B. Koo, Esq., Squire, Sanders & Dempsey L.L.P., 14th Floor, 801 S. Figueroa Street, Los Angeles, CA, 90017-5554, US

(21) Appl. No.: 09/843,508

(22) Filed: Apr. 26, 2001

(51) Int. Cl. A61K 31/198

(52) U.S. Cl. 514/562

(57) ABSTRACT

A method for treating and reversing the damages caused by anoxic encephalopathy and ischemic events is disclosed. The method according to the present invention comprises the administration of a therapeutically effective dose of N-acetylcystein.

### FIELD OF THE INVENTION

The present invention generally relates to a method for treating anoxic encephalopathy conditions and ischemic events.

### BACKGROUND OF THE INVENTION

Serious cerebral damages may result from an interruption of the blood flow to the brain tissues. The cerebral vascular syndrome or stroke is caused by a permanent obstruction of the blood supply into a limited area of the brain resulting in a complete loss of function of the affected area. This cerebral vascular syndrome or stroke can be generally characterized as an ischemic event.

An interruption of the total blood supply to both hemispheres of the brain, even temporary, may cause an irreversible damage. Such irreversible damage often leaves the patient in a vegetative state or coma with a complete loss of memory, speech, and other intellectual functions, even after the blood supply resumes its flow into the brain tissue. This syndrome is known as anoxic encephalopathy and it is one of the most devastating diseases known to man. In spite of many decades of research, no treatment is known for anoxic encephalopathy and the medical community has concentrated on preventative efforts only.

One of the main causes for the total interruption of the blood supply to the brain is an irregular heart rhythm. The most commonly known irregular heart rhythm is cardiac arrest. Even though major advances in the preventative treatment of cardiac arrest have been achieved over the last two decades, more than 100,000 new cases of anoxic encephalopathy occur every year in the United States.

At present, patients affected by anoxic encephalopathy and ischemia are left with no meaningful treatments to reverse the cerebral and associated neurological and functional damages that are caused by the interruption of the blood and oxygen supply to the brain. Therefore, there is a need for a treatment of the anoxic encephalopathy and ischemic conditions.

### SUMMARY OF THE INVENTION

The invention described herein is a novel method for treating and reversing the damages caused by anoxic encephalopathy and ischemic events. The method according to the present invention comprises the administration of a therapeutically effective dose of N-acetyl cystein, a drug having a previously unknown property to reverse damages caused by anoxic encephalopathy and ischemic events.

### DESCRIPTION OF THE INVENTION

N-acetylcystein is the N-acetyl derivative of L-cystein. The empirical formula of N-acetylcystein is C₅H₉NO₅S and its molecular weight is 163.191. Its structural formula is:

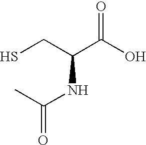

N-acetylcystein is known to have a potent antioxidant activity and has been primarily used in the treatment of acatylaminophen (Tylenol) overdose toxicity. Its mucolytic property has been frequently used in the treatment of bronchitis and other respiratory conditions.

Use of N-acetylcystein in the treatment of damages caused by anoxic encephalopathy and ischemic events has been previously unknown. According to the present invention, administering a therapeutically effective amount of N-acetylcystein to patients can reverse the cerebral and associated neurological and functional damages that are caused by the interruption of the blood and oxygen supply to the brain.

The useful therapeutic property of N-acetylcystein for treating patients affected by anoxic encephalopathy and ishemic events is demonstrated by the following clinical case reports.

Case No. 1:

An 87-year-old lady was admitted to the intensive care unit in a deep coma. Several hours prior to the admission, she suffered from a persistent ventricular arrhythmia that led to a cardiac arrest. She required nine successive electroshocks in order to reestablish her regular cardiac rhythm. The total interruption time of her blood supply to the brain was considered to be more than ten minutes. By the time her cardiac rhythm returned to normal, she was in a deep coma and did not respond to any painful stimuli. Her pupils were fixed and dilated, and she was placed on a mechanical ventilation. As she was unable to trigger the ventilator by herself, she was placed on an automatic mode of mechanical ventilation. There was no improvement in her condition over the next forty-eight hours. A neurologist determined her condition to be irreversible and hopeless. A second neurologist concurred. During the forty-eight hour interval, the patient was given 600 mg of N-acetylcystein in a twenty percent solution, administered via a nasal-gastric tube every twelve hours. During this forty-eight hour period, the patient completely revived from her coma. She awakened and was able to communicate by head nods and facial expressions. Her breathing mechanism resumed and she was able to breath partially on her own without the respirator.

Case No. 2

A 60-year-old high school teacher went into a cardiac arrest while driving in her car. By the time she arrived at a hospital emergency room and was resuscitated, a period of twenty to thirty minutes of an interruption of the blood supply to the brain had elapsed. Even though the cardiac resuscitation efforts were successful, she remained in a deep coma, a vegetative state, and required a life supporting mechanical ventilation. Over the next two years, she remained on mechanical ventilation, gastrostomy tube feedings, and in a coma without any improvement in her cerebral or neurological functions. Her prognosis for recovery was considered hopeless.

Approximately two years after the cardiac arrest, she was administered 600 mg of N-acetylcystein in a twenty percent solution twice daily. Over the next several months, she made a remarkable recovery. She awakened from her coma. She became alert and able to respond initially to tacit and aural stimuli, and later she was able to understand simple sentences. By the fourth month of treatment, she was able to respond verbally, initially with monosyllabic words, later with polysyllabic words, and then short sentences. She no longer needed the respirator.

By the fifth month, she was discharged from the sub acute unit where she had resided for two years and was transferred to the regular floor in an extended care facility. Even though she still retained a considerable neurological deficit, she continued to progress in her intellectual and motor functions, a state which was unthinkable five months before.

Case No. 3:

A 52-year-old man lapsed into a coma from a cerebral hemorrhage secondary to a vascular stroke. As his condition continued to deteriorate, he required a life supporting mechanical ventilation. Several months later he developed a brain abscess that required a craniotomy. This was followed by the appearance of an intense seizure disorder.

Following the surgery, it became possible to gradually wean him from the respirator, but he remained in a semi-vegetative state, unable to respond to aural stimuli and fed by a gastrostomy tube. He remained completely bedridden.

More than three years after the initial event, he was administered 600 mg of N-acetylcystein in a twenty percent solution twice daily via his gastrostomy tube. Over the next several months, he made a remarkable recovery. He became completely alert and able to respond to simple verbal commands. By the third month of treatment, he was able to sit in a wheelchair and express his wishes to be driven around the facility where he resided. By the fourth month of the treatment, he regained the capacity to express himself verbally even though it was restricted to a vocabulary of mono and disyllabic words. He also regained the ability to lift his head, which i previously lay flexed upon his chest, without the use of an assisting device. By the fifth month of treatment, he was able to ambulate more than one hundred steps with the aid of a walker or the wheelchair. His neurological and cerebral recoveries continued to progress.

Case No. 4:

A 42-year-old medical technician experienced a series of multiple strokes that led to a semi-comatose state lasting more than two years. Her medical condition deteriorated over time with the development of multi-organ failures. She required a mechanical ventilation and hemo-dialysis for life support. Three years after the onset of her condition and several months after all efforts to wean her from the respirator failed, she was initially administered 600 mg of N-acetylcystein in a twenty percent solution twice daily via a gastrostomy tube. The dose was progressively increased to 2400 mg daily given in 600 mg doses every six hours. She then improved and no longer needed the respirator.

Even though the exact biochemical mode of action of N-acetyl cystein on brain tissues at the cellular level is still unknown, the above cases indicate the therapeutic efficacy of N-acetyl cystein in reversing a coma as well as other neurological deficits associated with anoxic encephalopothy and ischemic events leading to the deprivation of oxygen in specific areas of the brain. The drug is able to reverse certain of these conditions, not only during the acute phase (i.e. several days following the brain damage), but also many months or years after the initial event.

The effective amount of N-acetyl cystein according to the present invention is not limited to the dosages described above, but may range from the daily dosage of 20 mg to 150 mg per kg of body weight depending on other factors such as the severity of the damage and the general physical conditions of a particular patient.

While the foregoing description defines an embodiment of the present invention, it is to be understood that it is subject to many modifications and changes without departing from the spirit and scope of the appended claims.

1. A method for treating a patient with anoxic encephalopathy conditions comprising administering to said patient a therapeutically effective amount of N-acetylcystein.

2. A method according to claim 1, wherein said therapeutically effective amount of N-acetylcystein is the effective amount necessary to reverse conditions caused by said anoxic encephalopathy.

3. A method according to claim 2, wherein said conditions comprise coma, cerebral and neurological damages, loss of memory and speech, and inability to walk.

4. A method according to claim 1, wherein said therapeutically effective amount of N-acetylcystein is a dosage between 600 mg to 2400 mg per day.

5. A method according to claim 1, wherein said therapeutically effective amount of N-acetylcystein is a daily dosage of 20 mg to 150 mg per kg of body weight.

6. A method according to claim 1, wherein said N-acetylcystein is in a twenty percent solution.

7. A method for treating a patient affected by an ischemic event comprising administering to said patient a therapeutically effective amount of N-acetylcystein.

8. A method according to claim 7, wherein said therapeutically effective amount of N-acetylcystein is the effective amount necessary to reverse and limit neurological and gangrene damages caused by said ischemic event.

9. A method according to claim 7, wherein said ischemic event is a peripheral vascular disease.
